(19) United States
(12) Patent Application Publication
(10) Pub. No.: US 2005/0038486 A1
(43) Pub. Date: Feb. 17, 2005

(54) Apparatus and method for producing facelift-like effects on soft tissue

(76) Inventor: Stephen Mulholland, Toronto (CA)

Correspondence Address: DENNIS G. LAPOINTE;LAPOINTE LAW GROUP, PL, 623 E. TARPON AVENUE, TARPON SPRINGS, FL, 34689, US

(21) Appl. No.: 10/639,390

(22) Filed: Aug. 12, 2003

(51) Int. Cl. A61N 1/18; E04G 1/00; E06C 5/32

(52) U.S. Cl. 607/48; 607/3

(57) ABSTRACT

Apparatus and method for non-surgical soft-tissue contouring and elevation of the facial envelope by strengthening the facial elevators of said soft tissue facial envelope. The method comprises inducing skeletal muscle hypertrophy by applying a series of applications of a tensile load to targeted facial envelope muscles while delivering a sufficient level of electrical current using electrode probes across said targeted muscles to facilitate contraction of the underlying muscles under the tensile load. The tensile load creates a concentric and eccentric load on the muscle fibers and muscle fiber hypertrophy is optimized. The apparatus includes an adjustable outrigger attached to the torso, elastomer bands attached to the outrigger and to fixation tabs on the facial envelope under tension. Optimally designed spherical-shaped probe tips are used to minimize localized burns at the margins of the radius and to eliminate current concentration points, while obtaining full contractile excursion of muscle fibers under stretch.

The invention relates to a non-surgical alternative to formal surgical facelift procedures using apparatus and an associated method for the vertical soft-tissue repositioning using hypertrophy of the deep musculo-facial layer by placing these muscles of facial elevation under tensile load conditions using electrical stimulation.

### BACKGROUND

The demand for non-surgical, non-ablative techniques of improving the signs and stigmata of aging has increased tremendously over recent years. Aesthetic medicine has witnessed the growth in medically driven skin care products for the improvement in epidermal and dermal skin architecture, microdermabrasion for the enhancement of stratum corneum, botulinim toxin for the treatment of hyperdynamic facial mimetic muscles and the rhytides they produce, injectable filler substances to smoothen fine lines, wrinkles and furrows, facial therapy and photorejuvenation treatments for the improvement of pink and brown discoloration, pores, skin texture and tone, ACNE blue-light emitting devices for the improvement in acne and laser hair removal systems for the permanent reduction and management of unwanted facial hair. All these techniques have facilitated and enhanced the ability to create more attractive, youthful, vital and often, beautiful appearing skin.

However, in most instances, the skin, although smoother and more even from these non-ablative skin enhancements, is still inferiorly displaced, droopy, sagging, loose and deflated. It would be tremendously appealing to aesthetic skin enhancement patients, if physicians could offer a procedure that delivered a predictable and noticeable degree of contouring and youthful vertical elevation of the soft-tissue envelope of the face without the need for surgery, a knife, pain, anesthesia, significant risk or recovery.

As we age, sun exposure, environmental damage and normal aging of the soft-tissue conspire to produce aged looking skin, including various proportions of telangiectasia, erythema, rosacea, hyperpigmentation, dyschromia, enlarged pores, fine and moderate wrinkles and furrows. The present invention preferably includes, in addition to its “lifting” component, adjunctive skin enhancement procedures that produce more youthful looking skin. As we age, there is also a deeper soft-tissue aging process involving atrophy of the fat and subcutaneous tissue, a senile myopathy with loss of skeletal muscle and an attenuation of dermal fascial attachments to this underlying atrophied musculafascial layer, resulting in an involution and inferior descent of the facial soft-tissue envelope. As the human body ages, the inventor herein found that in the absence of resistance training, the natural senile myopathy process results in 30-50% atrophy of skeletal muscle mass by the age of 50 years old, including the muscles of facial elevation. By the age of 70 years old, there has been up to 70% loss of skeletal facial muscle mass.

The theory behind the present invention is the reversal of this senile myopathy process, that is, through a resistance procedure, one can induce a facial myofibril hypertrophy with up to 50% increase in facial myofibril size and volume over an approximate 10 week, multiple visit program.

Introduction and Basic Science: As noted above, the present invention is a non-surgical alternative to formal surgical facelift procedures. As the human face ages, it suffers from a loss of fat and a senile myopathy characterized by a significant loss of skeletal muscle mass, strength and resting tone. The present invention is a coordinated series of several treatments, approximately 20, over approximately 10 weeks, with the vehicle for the vertical soft-tissue repositioning being hypertrophy (enlargement and strengthening) of the deep musculo-facial layer.

In experimental use on patients, the muscles of facial elevation typically underwent twice weekly resistance treatments, incorporating an anatomically precise high voltage electrical current application system and a resistive tensile loading device that results in isotonic, concentric and eccentric myofibril hypertrophy training of the muscles of facial elevation. The objective over the treatment period was a 30-50% hypertrophy of the facial muscle elevators, which, in turn results in a translational volumetric alteration of the muscle and a vertical elevation of the soft-tissue overlying these hypertrophied elevator muscles if the cutaneous envelope was still attached to the underlying musculature. During the program, patients also underwent other adjunctive skin renewal therapies designed to enhance the appearance of the soft-tissue envelope while undergoing the elevator therapy. These adjunctive skin enhancement treatments included ultrasonic microdermabrasion sessions, transdermal ultrasound assisted dermatophoretic skin care product delivery, intense pulse light and radio frequency therapy, and skin care product use. The protocol involved 20 treatments, of graduated increasing electrical current and tensile load, applied twice weekly. At the beginning of the 5th week, in a majority of consenting patients, all the active facial depressor muscles and muscles of excessive facial animation and wrinkles, were deactivated with botox and, at the end of the 9th week, injectable filler substances were used to augment the upper and lower lips and smoothen any residual furrows and lines uncorrected by the process. Patients were not candidates for the process if their skin was too loose and not attached to the underlying facial muscle layer or, there was too thick a layer of subcutaneous facial fat. In the study protocol, 103 consecutive patients underwent the lift procedure. All patients were evaluated, underwent informed consent and had preoperative photographic documentation. At the end of the study, a patient based survey was completed and post-procedural photographs taken. The age range in the study was 32-56 years old, with an average age of 46 years old. All study patients were female. Using a 10 point linear analogue scale of degree of improvement, the average brow and upper lid lift improvement was 6.3/10, with an average increase in the measurable ciliary lash line to supra-tarsal fold distance of 4 mm. The average degree of cheek lift and neck lift enhancement was over 50% improvement. The patient based perception scores showed an overall “years of rejuvenation achieved” of 5.7 years younger looking following the procedure. The overall patient happiness with the procedure was 88%. Over 80% of the patients continued with the recommended monthly booster lift maintenance program.

It was concluded that combination skin enhancement therapies and hypertrophic resistance MyoFacial procedures, as disclosed herein, can simulate many of the effects of surgical brow, face and necklift procedures and can significantly restore a more youthful, healthy and “lifted” look without surgery.

Candidates for the present inventive method typically would have mild to moderate facial soft-tissue descent, including: (i) brow ptosis, with excess skin of the upper lid, (ii) cheek and jowl ptosis with lower lid malar-palpebral grooves, increased lower lid-cheek distance, deepened smile lines, commissural descent and early jowls, and (iii) cervical laxity, platysmal bands and loss of an acute cervical angle. As noted above, the process is an alternative to a surgical procedure and will simulate many of the effects of an operative intervention (perhaps 30% of what surgery might deliver). However, the procedure is not a substitute for a facelift, brow or necklift surgery and if the patient desires the best possible outcome, regardless of risk, recovery or expense, they should always be offered the cosmetic surgical procedure.

The principles of this inventive hypertrophy and resistive training of the face are as follows:
1. A tensile load must be applied to the targeted muscle of facial elevation and all the myofibrils and myofilaments should be in full stretch.
2. The tensile force during a lift procedure is provided by the resistive apparatus.
3. Maximal hypertrophy of skeletal muscle fibers occurs with optimal tension, limited repetitions and is repeated over several sets.
4. Optimal power enhancement and myofilamentous hypertrophy occurs with 8-12 repetitions over 3-6 sets.
5. At the end of 20 visits and 10 weeks (twice weekly for an hour), the inventive procedure will provide a strengthened and hypertrophied musculo-soft-tissue diathesis and a noticeable elevation of the attached soft tissue envelope.

There are two basic types of hypertrophy procedures. Just as when you join a gym and you begin a weight lifting program, you would start with very light weight and very little resistance (tensile force). Too much resistive tensile load during the first week of the weight training program would likely result in muscle damage, micro-trauma, lactic acid buildup, pain and stiffness. However, after 2-3 weeks of training with light weights, your muscles have begun the hypertrophy process and increasingly heavy weight can be applied with less likelihood of injury to the individual muscle fibers. Optimal strength increases and hypertrophy occurs over the first 10-12 weeks, with few repetitions and maximal tensile force applied to muscle fibers stretched to their fullest length. Exactly the same thing is accomplished with the present invention. The process is started with little resistance on the facial muscles of elevation and then gradually add tensile force in an isotonic, eccentric, concentric fashion to optimize hypertrophy.

There are preferably three phases to treatment of a patient: (i) the non-resistive process; (ii) the isotonic resistive hypertrophy process; and (iii) maintenance processes, including the isotonic resistive hypertrophy process. This present invention relates to the isotonic resistive hypertrophy processes.

Non-Resistive Hypertrophy Process

The non-resistive process is performed during the first 2-3 weeks (first 4-6 sessions). There is no resistive apparatus used and thus, no tensile force on the myofilaments. During these non-resistive treatments, there is strong electrical stimulation and contraction of the facial musculature. The lack of resistance allows the patient to become acclimated to the therapy without undue injury to the facial muscles of soft-tissue elevation. The treatments are performed twice weekly, each for 45 minutes and the first 4-6 sessions are performed without the resistive apparatus.

Resistive Hypertrophy Process

After the first 3 weeks (6 sessions), the Resistor is applied to the patient. Twice weekly, 45-minute resistance training sessions are then performed for the next 8 weeks with isotonic, eccentric and concentric contractions of the facial muscles of elevation and youth.

Maintenance Processes

After completing the full course of treatments over the approximate 10 weeks, the patient should commit to a maintenance program to maintain the hypertrophy of the facial elevator muscles. As the intensive 10 week in office treatment program was designed to create the soft-tissue facelift effect, the intensity of the maintenance treatments need not be as aggressive to preserve the facial elevation.

In Office Maintenance Treatments

To maintain the facial elevator muscle myohypertrophy booster treatments are recommended every 4-6 weeks, depending upon the thickness of the integument and strength of the muscle.

As an aid to better understanding the process of the present invention, a detailed description of the non-resistive process is provided.

The Non-Resistive Technique

1. The patient's hair should be protected with a band or O.R. cap. Towels should be placed around the patient's collar
2. The patient's face should be cleansed with a mild alcohol based skin cleanser, removing any oil and dirt from the skin and blot the facial skin dry.
3. Put fresh water in a bowl a side table and have gauze and ultrasonic gel near the power generating equipment and monitoring equipment (control panel/system).
4. It is preferred that the physician or technician have sufficient tinted (such as blue tinted) conductive gel in a clear plastic 6 oz cup and a tongue depressor.
5. The equipment should be turned on and operational parameters should be programmed.
6. Initiate the generation of the premodulated waveform, including the settings for allowing the necessary sinusoidal, premodulated wave pattern of the process. The premodulated waveform should be set to “continuous”.
7. Preferably a timer should be set to count in seconds.
8. Place both electrodes in the gel, approximately 1 inch apart and so that half of the shaft of each probe is submerged in the gel. Make sure the probes do not touch each other or the sides of the cup as this contact will deactivate the electrical charge required for treatment.
9. With the probes in the gel, program the current by adjusting the current to the desired amperage. Allow the current to reach the desired level. The current level used will vary depending upon the tolerance of the patient, the specific facial muscle elevator being treated and how many weeks into the program the patient is. The following current levels are good starting points, from which you can adjust specific levels:
   Frontalis=25 mA (milliAmpres)
   Orbicularis=20 mA
   Zygomaticus=30 mA
   Digastric=35 mA
   Platysma=40 mA
10. With the desired current entered for the treatment of the particular facial region, remove the probes from the gel in a scooping fashion. Ensure that there is a small collection of gel on the end of each probe. There must be enough gel on the skin at the probe-skin interface or there may be excessive epidermal resistance to the passage of electrical current and localized pain or a burn may ensue.
11. While keeping the probes separate from each other, check once again the desired current level and adjust if necessary.
12. The technician is now ready to begin the hypertrophy treatments.
   As previously mentioned, the first 4-6 sessions, over the first 2-3 weeks are typically performed without the resistive device.
13. Electrical current levels are determined by patient sensitivity. It is recommended that the technician always begin at lower amperage levels (18-25 mA) and then increase the voltage up toward and then above the guidelines above.
   Muscle Elevators
   Frontalis Hypertrophy

The Frontalis is a broad band striated muscle that attaches through dermal fascial attachments of the frontal brow ridge. The origin of the frontalis is the through the galeaaponeurotica, posteriorly to the occupitalis. The frontalis is the prime elevator of the brow. It is the lateral frontalis that is usually the prime target, for it is the lateral brow which tends toward ptosis creating excess upper lid skin and a sad or tired appearance. Most patients will desire a high lateral arch of the eyebrow complex, which will highlight the beautiful light reflex of the lateral orbital rim. During the consultation process, the technician will have worked with the patient to determine the optimal, aesthetically pleasing brow orientation and elevation. Some patients may desire a central brow elevation to round out their brows and, rarely, some with central depressed brows from corregator supracilii and depressor supracilii over activity may desire a more medial brow elevation to balance the pre-existing lateral brow elevation. During the treatments, the technician will have an opportunity to isolate and target the segment(s) of the frontalis for which the technician may provide the optimal elevation. The desired segment of the frontalis is isolated from which the technician would like to obtain the aesthetic elevation.

With adequate gel on the electrodes, the probes are place on the skin. Starting with the inferior probe, closest to the orbital rim, the apex of the round head of the probe with its gel is placed on the orbital rim, just over the eyebrow hair. Then the second probe is placed on the superior aspect of the brow and frontalis, just inferior to the hairline. This 1-2 placement of the probes will help prepare patients for the impending electrical current. The patient should be warned that as this superior probe touches the skin, there will be a discomfort, resembling “tugging” on the hairline, or a pulling of the hair. This represents the electrical current surging through the frontalis muscle and frontotemporal branches of the facial nerve and stimulating nerve endings from the Supraorbital and Supratrochlear nerves.

When both probes are touching the patient's skin, the preset level of electrical current will be passed through the gel, through the skin, along the facial nerves and then the Sarcolemma of the frontalis muscle and stimulate an action potential of the fronto-temporal nerve branches of the facial nerve. The alternating, cyclic contractions of the frontalis muscle will be noticed. The control system is programmed to provide a premodulated alternating current of an effective waveform and current density which will result in an automatic contraction, relaxation cycle for the facial muscle being stimulated.

The initial few contractions will not elevate the brow optimally. With each subsequent contraction, there is recruitment of more myofilaments and the muscle fiber length and volume that is contracting increases. After the first few contractions, there should be a full 1-2 cm excursion of the frontalis, as witnessed by the overlying skin movement. If the patient is tolerating the electrical current setting, the probes can be moved back into the gel, without them touching and the current can increase by 5 mA increments, for repeating the trial contractions of the frontalis. The stimulation process should be repeated, this time, allowing 8-10 full contractions (this will be your first frontalis set). After the 8-10 contractions, the probes should be returned to the gel and the current raised to 30 mA. The probes can then be returned to their positions on the forehead, using the 1-2 inferior to superior sequence outlined above. If this current level is tolerated for the next set of 8-10 contractions, then the current is raised to 40 mA. The process is continued at 40 mA (if tolerated by the patient) with 8-10 repetitions.

After each set of 8-10 contractions, the probes are again returned to the gel container for new gel. This process is repeated until 4-6 sets (total 40-60 contractions of the lateral frontalis muscle) have been completed. If the patient is unable to tolerate the 40 mA current, because of discomfort, then, with the probes in the gel, the current should be returned to a patient acceptable level to complete the requisite number of 4-6 resistance training sets of 8-10 repetitions.

Horizontal Orbicularis Hypertrophy

As we age, there are several distinct anatomic and physiological changes to the eyelids that contribute to a more aged appearance.
(i) The orbicularis-tarsal support sling of the lower lid becomes lax, with attenuation of the ligamentous attachments of the lateral canthal tendons to the orbital rim and generalized skeletal muscle atrophy. This attenuation of the orbicularis-tarsal sling can result in increased scleral show and shortening of the interpalpebral distance.
(ii) Generalized atrophy of skeletal muscle, including the Orbicularis occuli and its septal supportive system can result in herniation of the intraorbital, extraconal fat, which in turn leads to lower lid “fat bags” and a tired, aged appearance to the periorbital region. Hypertrophy of the orbicularis sling will strength the supportive function of this muscle and minimize the herniation of the lower lid.
(iii) As we age, certain individuals, especially in skin type 1-3 individuals, there occurs a venous engorgement of the deep reticular dermis and the development of deep, draining subcutaneous reticular veins. This complex lower lid venous congestion leads to the appearance of aged looking dark circles in a large number of Caucasian skin types. The treatment or process brings enhanced dermal blood flow to the lower lid complex, facilitating increased drainage of the lower lid venous engorgement and improvement in the appearance of the lower lid “dark circles.”
(iv) The aging process of the skin, both solar-actinic damage and nonphoto-aging, results in an alteration of the normal appearing collagen and elastin content of the lower lid skin. Loss of collagen, ground substance and the interstial interadermal water associated with these complex sugars results in atrophy of the dermis and rhytides. Excessive animation of the orbicularis occuli can create hyperdynamic rhytides in the crows feet region of the lateral orbicularis complex and a lower lid orbicularis hypertrophy and roll, as well as crepe loose lower lid skin.
(v) Generalized atrophy of the orbicularis occulii may contribute to herniation of the upper lid medial and central extraconal, intraorbital fat pads.

A botox technique may be used in conjunction in these latter areas of the face. One of the 5-6 facial regions where the facial muscles act as depressors that pull down age the face, are the lateral and vertical fibers of orbicularis occuli. Botox treatment is utilized in the vertical components of the lateral orbicularis occulii. The lateral orbicularis occulii hyperdynamic activity contributes to the aged periorbital in the following ways:
(i) The lateral, vertical orbicularis occulii are the prime depressors of the lateral brow and recurrent contraction and hyperactivity over many years contributes to ptosis of the lateral brow and excess upper lid skin and tissue. Botox in this region will allow, through paralysis of the strong depressor activity of the lateral orbicularis an unopposed action of the lateral frontalis and a moderate 1-2 mm chemical elevation of the lateral brow, lifting some of the aging flesh off lateral upper eyelid. This elevation effect of the botox technique is then greatly amplified by the hypertrophic treatment of the lateral frontalis and often another 2-8 mm of lateral brow elevation can be achieved.
(ii) The orbicularis occulii hyperdynamic mimetic muscle may lead to exaggerated, excessive crows feet and crepe loose lower lid skin, a lower lid orbicularis hypertrophy roll and rhytides. Botox in this region will minimize rhytides and the lower lid orbicularis roll. The use of botox to ameliorate many of the lateral, lower lid Orbicularis hypertrophic effects allows the technician to work aggressively on the orbicularis to tighten the lower lid sling effect without contributing to some of the hyperrhytidogenic effects of the lateral orbicularis on the crow's feet. If botox where not applied to the lateral orbicularis region, the contractions in this region could, conceivably, worsen the crows feet rhytides.

In treating this area, the probes are placed in the gel container. The current is set to 25 mA. The inferior probe, with adequate gel, is placed on the orbital rim just below the lateral canthal tendon. The second probe is placed at the level of the medial canthal tendon on the orbital rim.

Allow for 8-10 contractions, with recruitment and skin excursion. Repeat this cycle two of three times, increasing the current while the probes are in the gel, until the current applied is between 25-45 mA and there is more than one centimeter of skin excursion witnessed. Once the desired excursion and current level has been reached, 4-6 sets of 8-10 contractions each are repeated at the highest current the patient can tolerate.

The Horizontal Orbicularis Hypertrophy will result in significant hypertrophy of this muscle and augmented myocutaneous blood flow and circulation. The improved blood flow may help with the dark circles of venous engorgement. Over the treatment period, the enhanced cutaneous blood flow and favorable oxygen tension gradients and characteristics that develop as a result of the process helps to contribute to increased dermal blood flow and the production of subtle dermal fibrous tissue and ground substance that there will be a minor to moderate enhancement in the appearance of fine rhytides of the lateral crow's feet.

Inferior Orbicularis Occuli Hypertrophy Treatment

Starting at 20 mA, one probe with its freshly procured gel is placed along the medial orbital rim and close to the nasal side wall.

The second gel dipped probe is placed close to the lateral orbital rim. Contact of this second probe with the skin with result in a very significant contraction of the entire orbicularis complex, but especially the lower lid orbicularis-tarsal sling. Allow for 8-10 contractions and then increase the current 5 mA at a time for each set of 10-12 contractions until 25-40 mA is reached. Complete 4-6 sets of 8-10 contractions each at 25-40 mA current and maximal orbicularis contraction and skin excursion (the eyelids should be forced shut with each contraction.)

The Inferior Orbicularis Occuli Hypertrophy will:

(i) Strengthen the lax horizontal sling and improve the appearance of herniation of upper and lower lid fat pad herniation.
(ii) In addition, the enhanced cutaneous blood flow will enhance dermal collagen and ground substance production, with some improvement in the lower lid crepe loose skin and rhytides.
(iii) Enhanced dermal blood flow will improve the venous congestion of the lower lid complex and lessen the dark circles that can develop.
   Zygomaticus Hypertrophy

The aging face is usually characterized by descent of the midface, with a ptosis of the cheek fat-pad and soft-tissue envelope. This midface descent results in an aged appearing elongation of the lower lid-cheek distance, exacerbation of the lower lid naso-jugular groove and malar-palpebral grooves. With descent of the cheek, one will see deepening of the nasolabial groove, descent of the commissure, appearance of labiomental lines and early pre-jowls all of which contribute to an aged appearing midface. The hypertrophy of the Zygomaticus major and minor muscles, strengthens and enlarges these muscles which, by way of their dermal attachments creates a vertical elevation of the attached skinfascial envelope and a more youthful cheek, diminished jowl and prejowl, with a tighter jawline. The use of the botox technique in the Depressor Angularis Oris, a prime depressor of the commissure of the mouth and one of the major midface depressor muscles will, when paralyzed with botox, will facilitate unopposed contraction of the midface elevators, the Zygomaticus complex. Hypertrophy of these facial elevators, with a paralyzed depressor complex, will amplify the vertical elevation effects. The Levator Labii Superioris will also undergo hypertrophy and, together with uncoupling botox in the lateral orbicularis oris, will “roll out” the lateral lip.

To treat this area, with the probes back in the gel container, set the current to 35 mA. Take the superior probe and place it, with adequate gel, on the cheek skin, just inferior to the mid-point of the malar prominence, but superior to the nasolabial fold. Take the other probe, scoop the necessary amount gel and place it superior to the inferiorly place probe, but superior to the malar eminence. With placement of the second probe, the technician will begin to see contraction of midface musculature. In addition, there will be electrical stimulation of the upper zygomaticofacial and frontotemporal branches of the facial nerve. The upper facial nerve branches will cause a synchronous contraction of the orbicularis occulii and frontalis. This moderate mass action of the upper face should not be a concern, as botox treatment will compensate. Concentrate on the migration of the modiolus, or common muscle attachment of the commissure. Slowly move both the malar Myofacial probes around a few millimeters in their respective places until significant, optimal vertical angular elevation of the modiolus and cheek is witnessed. With adequate positioning of the electrodes and sufficient current, the treatment should result in a 2 cm elevation of the modiolus and corner of the mouth, in an oblique angle up towards the top of the ear.

After 8-10 contractions of the zygomaticus major and minor. The probes should be placed back in the gel container and the current increased to 45 mA, in 5-10 mA increments, scooping more gel onto the probes and relocated back on the optimal zygomaticus position on the malar prominence, facilitating optimal zygomaticus excursion and contraction.

4-6 sets of full excursion zygomaticus contractions should be completed, with each set containing 8-10 contraction repetitions in each set. Some patients will have periapical dental disease that will make the electrical stimulation of the lower zygomaticus musculature sensitive, especially over the canine tooth and premolars. With these patients, the technician will have to move the probes more superiorly to avoid excessive dental sensitivity and still achieve zygomaticus stimulation. Additionally, the technician may have the patient place his or her tongue in front of the sensitive tooth during treatment.

Orbicularis Oris Hypertrophy

Hypertrophy of the Orbicularis Oris results in augmented Myocutaneous blood flow to the dermis. This augmented blood flow has an ultrastructural effect upon the quality of the dermis. There is often a softening of the perioral, vertical rhytides (bleed lines) and a subtle augmentation of the white roll of the vermillion border. In addition, there is an age related atrophy of all skeletal muscles, including the muscles of facial animation. With significant loss of muscle volume and mass, there can be an “accordion” effect of the perioral skin collapsing on itself forming vertical upper and lower lip lines. Therapy will result in a hypertrophy of the orbicularis oris and a “plumping” effect on the overlying creases and a softening of the perioral rhytides.

To treat this area, start with the electrodes inside the gel cup; set the current to 20-25 mA; place the heads of both electrodes at the level of the vermillion border, on either side of the philthral columns; and move slowly laterally to the commissures. Once both electrodes are on either side of the mouth, there will be a contraction of the orbicularis oris, forming a pucker of the lips. The technician then gradually replaces the electrodes in the ultrasonic or conductive gel cup, as described in the previous sections and increases the voltage until the mA is in the range of 30-40 mA and a 1-2 cm excursion of the lips into a full pucker is witnessed. Once again, 4-6 sets of 8-10 contractions each are performed.

Patients with excessive orbicularis oris hypertrophy and hyperdynamic ruggae (deep furrows of the lip) will be subject to botox treatment in the perioral region. With the softening effect of the botox, the enhanced blood flow effects of the hypertrophy treatment is still desirable and the perioral therapy is still performed.

Digastric-MyoHyoid Sling Hypertrophy

A prominent feature of the aging face and neck is a loss of the cervicosubmental angle. An acute cervical angle looks youthful, strong and attractive. This acute neck angle is often replaced by a more oblique, obtuse aged cervico-submental angle. The loss of the strong neck and jawline contributes significantly to the aging appearance of the face and neck. Hypertrophy of the Digastric muscle (Anterior and Posterior bellies that span the posterior skull base, attaching via the hyoid to the undersurface of the central symphysis of the mandible) and the Mylohyoid (which is the diaphragm of the floor of the mouth and spans from one side of the anterior mandible to the other) will help produce youthful contour changes of the cervico-submental angle. Hypertrophy of these two muscles results in an elevation of the hyoid bone and a re-draping of the soft-tissue envelope in the more acute contour. Submental lipodystrophy will compromise the visual result that can be obtained and submental liposuction may be needed prior to the lift for the optimal desired result.

To treat this area, with the probes in the ultrasonic (conductive) gel, set the current to 30 mA. Place one probe at the center of the submentum, 1-2 cms posterior to the central mandibular gonion. Place the second probe at the posterior aspect of the posterior belly of the digastic, towards the styloid process at the base of the skull. Watch for contractions of the digastic-mylohyoid complex. Visible elevation of the Hyoid and cervical angle superiorly and a contraction of the Mylohyoid should be witnessed. A bulge in the mid-neck represents the Hyoid migration with each contraction. There will often be a concomitant depression of the depressor angularis oris. This synchronous stimulation of the depressor of the commissure is not a concern. For these pre-jowl patients, this Depressor Angularis Oris will be paralyzed with the botox technique. The contractile process is continued, gradually raising the current to 40-45 mA as tolerated.

Once optimal voltage and contraction has been reached, perform 4-6 sets of 8-10 contractions of the digastric-mylohyoid complex with optimized current and clinical evidence of contraction and hyoid elevation.

Platysmal Hypertrophy

Loss of Platysmal tone often contributes significantly to the appearance of aging neck in the following ways:
(i) Vertical laxity of the overlying soft-tissue envelope of the neck and visible vertical skin laxity.
(ii) Loss of lateral platysmal tone, weakens the musculo-fascial sling that helps to support the submandibular glands in their youthful, hidden submandibular location. With age and a loss of platysmal tone, there is an exaggerated ptosis of the glands and the familiar bulge of the ptotic gland, under the pre-jowl in the submandibular region, all of which contributes to the loss of the youthful facial Ogee, or oval shape to the more aged droopy, bottomed-out, square lower face shape.
(iii) Loss of medial platysmal tone and redundancy often leads to redundancy of the medial platysma and platysmal bands or cervical cords.
   With Hypertrophy of the platysmal muscle, there is:
(i) a tightening of the submandibular gland support, minimizing their visibility;
(ii) a tightening of the cervical skin, improvement in cutaneous flap blood flow characteristics with concomitant skin rejuvenation;
(iii) improvement in the texture and tone; and
(iv) with hypertrophy of the platysma, a tightening of the medial platysma and a botox mediated lessening of the visible cords and bands.

With significant medial platysmal bands/cords, botox is used in the medial platysmal cord, to soften their appearance and the process is continued.

To treat this area, the technician places one electrode, with adequate gel, on the lateral platysma on one side of the neck at the approximate level of the hyoid. With the voltage set on 30 mA, the technician places the inferior electrode, with an adequate amount of gel, on the platysmal muscle, just above the clavicle and in line with the electrode above it and starts a series of contractions. The technician then relocates the electrodes in the gel and turns up the voltage until 40-45 mA is reached and a significant 1-2 cm of cervical skin elevation with each contraction should be observed.

4-6 sets of 8-10 contractions each at these voltages should be performed. Once the technician has completed one half of the face and neck, it is useful and encouraging to the patient during the first few sessions, to sit the patient up and point out to them in the mirror, the noticeable the youthful facial asymmetry that has been created with the hemifacial process, with the treated side showing a visibly elevated brow, cheek, tightened jawline and neck. This is especially true when the resistive apparatus, as further discussed hereinafter, has been employed after the 3rd week. This 1/2 face treatment technique is also a valuable consultation closure technique. Thus, after completing the hemi-face and before moving over to the other side, it is useful and encouraging to show the effects of the unilateral treatment to the patient using a handheld mirror.

The exact same sequence is performed on the opposite side of the face. Each side of the face requires approximately 20 minutes of treatment time for a total of 40 minutes for the process to be performed.

Resistive Hypertrophy Technique

After the non-resistive treatments, most patients have accommodated to the sensation of high voltage electrical current coursing through their faces, facial musculature contracting vigorously and involuntary anatomic facial part excursion. By the beginning of the third or fourth week of an approximate 10 week program (at the start of the 5th or 7th session, depending upon the week started), the present invention resistive apparatus is applied. The base of the resistor chest piece is secured for treatment by strapping it around the patient's body, typically by using attached hook and loop fasteners such as that made by Velcro Industries, B.V. LLC. Once the base is secured to the patient, the appropriate MyoFacial Outrigger, which can be in the form of a roll, is attached to the center of the base, again preferably using hook and loop fasteners. There are two different myofacial outriggers, one for the face and the other for the neck treatments, the difference being that the one for the face is spaced-apart more from the chest piece. The end of the outrigger roll with the metallic outrigger and the resistance bands faces the patient's face. The bands are then fastened to the resistive positions for the brow, midface and neck using electrode tabs. Preferably, these tabs similar to EKG electrode tabs should further be secured to the skin with Mepore™ or Hypafix™ medical tape. The resistive bands put a counter-traction on the facial skin and the muscles of facial elevation.

Resistive Hypertrophy Positions

1. Frontalis Position

The lateral frontalis undergoes hypertrophy after applying the resistive apparatus to the medial brow. The following steps are taken to engage the resistor.
(i) Attach the electrode to the medial brow.
(ii) Stretch the resistive band from that side of the metal outrigger to the electrode tab and hold the elastic in place with precut medical adhesive tape.
(iii) There should be sufficient tension on the resistor band such that the band is taught and the local skin is pulled down towards the resistor. Again, the elastic should be taught and the local skin under the electrode patch should be pulled down and slightly towards the resistor to ensure that there will be sufficient tensile load on the musculature and optimize the eccentric, concentric principles of the isotonic hypertrophy process. Once again estimate the degree of stretch, hence tensile load that is placed upon the facial muscles by inferiorly displacing the brow or modiolus.
(iv) Proceed with 4-6 sets of 8-10 contractions at the voltage parameters used during the non-resistive sessions. Utilize the highest electrical voltage that the patient can tolerate and which can be gradually built up towards during the non-resistive phase.
   2. Midface Orbicularis Occulii and Zygomaticus Position

Prior to beginning the orbicularis occulii and zygomaticus major and minor, attach the resistor to the midface position:
(i) Attach the electrode patch to the modiolus at the commissure.
(ii) Stretch the resistive band onto the electrode and hold it securely in place using the medical adhesive tape.
(iii) Again, the band should be taught and the local skin should be pulled down by the resistor to ensure that there will be sufficient tensile load on the Midface Zygomaticus musculature and optimize the eccentric, concentric principles of the isotonic hypertrophy process.
(iv) Proceed with 4-6 sets of 8-10 contractions in each set. Utilize the highest electrical voltage that the patient can tolerate and which can be gradually built up towards during the non-resistive phase.
   3. Cervical Resistor Position

Prior to beginning the Digastric-Mylohyoid and Platysmal hypertrophy sets, position the resistor in the cervical position.
(i) Change the Resistor Outrigger Roll to the smaller diameter, cervical model.
(ii) Attach the electrode patch to the base of the neck, just above the clavicle.
(iii) Stretch the Resistor band from the metallic resistor outrigger to the electrode and hold securely with medical adhesive tape. Again, the elastic should be taught and the local skin should be pulled down and towards the resistor to ensure that there will be sufficient tensile load on the cervical platysmal musculature and optimize the eccentric, concentric principles of the isotonic hypertrophy process.
(iv) Proceed with 4-6 sets of 8-10 contractions in each. Utilize the highest electrical voltage that the patient can tolerate and which can be gradually built up towards during the non-resistive phase.
   Care After the Treatment

After completion of the hypertrophic therapy for each of the facial regions, brow, lids, midface, perioral region and neck. Scrape the gel from the patient's face and discard. Squeeze excess water from the gauze in the bowl and wipe over completed areas to remove any residual gel. By removing the gel, you will help prevent the patient's skin from becoming uncomfortably itchy as the gel dries on the skin. Once the treatment is done, unused gel is protected by a film of saran wrap and used gel is discarded.

Once the isotonic hypertrophy therapy is completed to all facial elevators, cleanse the facial skin with a deep pore cleansing agent and apply hydrating, anti-oxidant and skin care products under an appropriate moisturizer and sunscreen.

Treatment parameters, number of contractions and sets should be entered on the patient record and office chart. Any unusual observations, comments or patient reactions should be recorded as well.

The above-described treatment regimens are intended to be exemplary only and not limited in scope.

### BRIEF DESCRIPTION OF THE DRAWINGS

In the accompanying drawings:

FIG. 1 is a kit style schematic representation of the components of the invention used to perform the resistive hypertrophy inventive treatment, wherein a typically configured smaller resistive outrigger is depicted for treatment of the neck area and a typically configured larger or taller resistive outrigger is depicted for treatment of the facial area;

FIG. 2a is a schematic representational depiction of a typical application using the larger resistor with one (for drawing simplicity sake) connective resistive elastomer band connected to a tab above the eyebrow and the probes being applied the treat the appropriate area of the face;

FIG. 2b is a schematic representational depiction of a typical application using the smaller resistor with one (for drawing simplicity sake) connective resistive elastomer band connected to a tab in the neck area and the probes being applied the treat the appropriate area of the neck;

FIG. 3a is a schematic depiction of the muscles of the face and neck that contribute to soft tissue elevation; and

FIG. 3b is a schematic depiction of the muscles of the face and neck that contribute to soft tissue depression and facial rhytides.

### DETAILED DESCRIPTION OF THE INVENTION

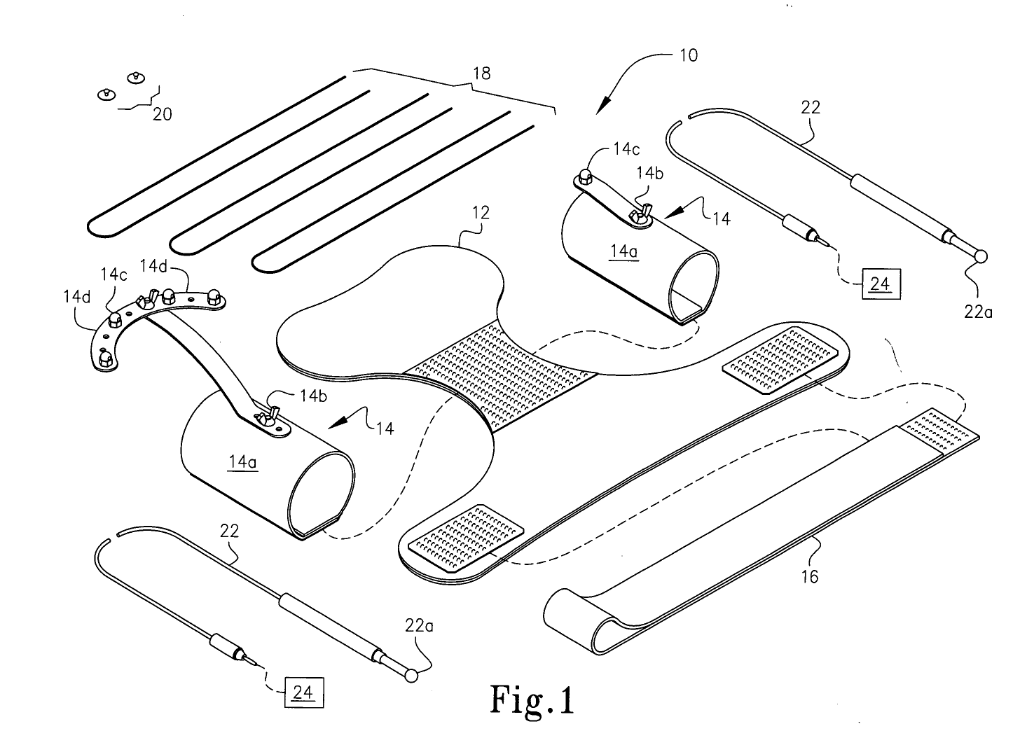

Referring now to the drawings, FIG. 1 representationally discloses in kit form, a practical application or example of components utilized in the present invention, and which is depicted generally as 10.

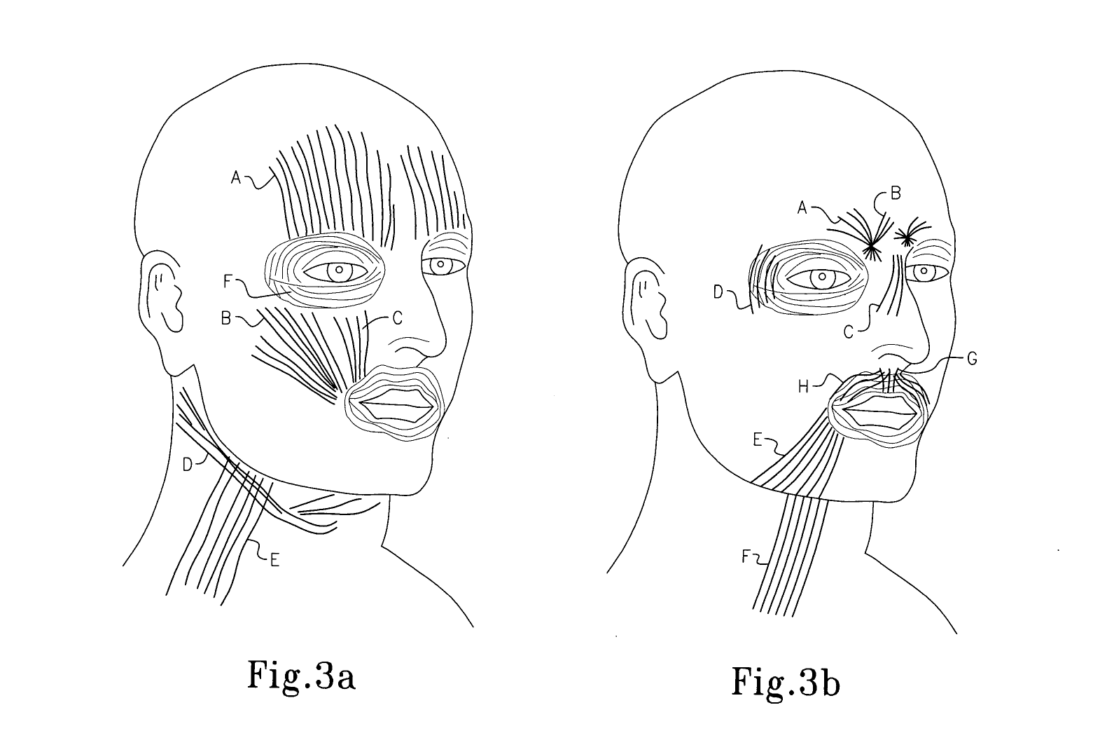

For a better understanding of the effected muscles targeted in the inventive process, FIG. 3a is a schematic depiction of the muscles of the face and neck that contribute to soft tissue elevation, that is, A. The Frontalis; B. The Zygomaticus Major and Minor; C Levator Labii Superioris; D. The Digastric sling; and E. The Lateral Posterior Fibers of the Platysma; and F. Horizontal Fibers of the lower lid Orbicularis Occulii. FIG. 3b is a schematic depiction of the muscles of the face and neck that contribute to soft tissue depression and facial rhytides, that is, A. The Corregator Supracilii and B. Depressor Suprcillii; C. The Procerus; D. The Lateral, vertical fibers of Orbicularis Occulii; E. Depressor Angularis Oris; F. The Medial Platysmal Bands; G.; The Depressor Septi Nasii muscle; and H. The lateral fibers of Orbicularis Oris.

To overcome the senile myopathy process and create significant myofibril hypertrophy of the facial elevators, the present invention was developed to accomplish the “lift.”
(i) Anatomic Precision: The only muscles we want to significantly stimulate and hypertrophy are those muscles responsible for elevation of facial soft-tissue. The Frontalis for the brow and upper lid, the Zygomaticus Major and Minor for elevation of the cheek-fat pad, commissure and pre-jowl, the Levator Labii Superioris for elevation of the lateral lip, the Digastric Sling for elevation of the hyoid sling and tightening of the cervical soft tissue envelope and lateral Platysma for the retention of the submandibular gland envelope. During the treatments, care is taken to avoid excessive contraction of those muscles that depress the facial soft-tissue or cause facial rhytides, the Depressor Corregator and Depressor Corregator Supracilii, Lateral Orbicularis Oculii, Lateral Orbicularis Oris, Depressor Septi Nasi, Depressor Angularis Oris and Medial Platysma. To accomplish this degree of precision, anatomic probes (see FIG. 4) were developed that facilitate transfer of high energy electrical current to the facial muscles of elevation without soft-tissue burns at the probe sites.
(ii) Premodulated Electrical Current WaveForm: A generator was developed to provide a premodulated waveform that delivers an adjustable high voltage current with a specific on/off sinusoidal waveform whose configuration is related to the average cross-sectional muscle volume of the facial strap muscles. The premodulated waveform allows the treating technician to isolate the desired facial elevator muscle and hold the probes in position, while the premodulated, pulsatile electrical waveform delivers the necessary pulsatile current waveform to facilitate the recommended 10 repetitions and 4 sets. The high voltage current density is required to overcome the tensile loaded muscles fibers and deliver a sarcomere-tosarcomere contraction.
(iii) Tensile Load: Without tensile load, there is no resistance and without resistance training, there is no rapid myofibril hypertrophy. The present invention 10 includes a resistor, which was developed to facilitate resistance training. In addition, a specific protocol was developed that delivers consistent lift effects and was delegable to non-physician staff within the physician's office.

In effect, each treatment is a resistance training workout for the muscles of facial elevation. It is recommended that each individual treatment take approximately 30 minutes to complete and require 4 sets of 10 repetitions for each facial muscle elevator, performed twice weekly for 10 weeks. The muscles of facial elevation that undergo the process every visit include the Frontalis, the Zygomaticus Major and Minor, the Levator Labii Superioris, the Digastric Sling and the Lateral Playtsma, and depending upon the patient, the horizontal sling support function of the Orbicularis Occuli and the Orbicularis Oris. These muscles of facial elevation must also overcome the tonicn and dynamic influences of the muscles of facial depression and rhytidogenic muscles.

More specifically the present invention is an apparatus 10 for producing facelift-like effects on the soft tissue facial envelope, including the brow, lids, midface, perioral region, jowls, cheeks and neck, by strengthening the facial elevators of said soft tissue facial envelope, the apparatus 10 includes a tensile resistive patient fixation structure or chest piece 12. The chest piece 12 lies on the front part of the torso and is therefore generally planar in shape, and may be formed with cutouts for a woman's breasts so that it lays in a relative flat orientation on the patient's chest. Included is a resistive outrigger 14 releasably attached to the fixation structure 12.

Means 16 for attaching the fixation structure 12 to the torso of the patient is provided. This can be in the form of a wrap around belt type of feature such as hook and loop fasteners made by Velcro Industries.

One or more resistive elastomer bands 18 and one or more resistive elastomer band fixation tabs 20 (or electrode tabs such as those used for EKG's) are provided. The tabs 20 are releasable attached to locations on the patient's soft-tissue to be treated. Each elastomer resistive band 18 is attached on one end to the resistive outrigger 14 and an opposite end of the elastomer resistive band 18 is attached to the fixation tab 20 in such a way as to create a tensile load condition measured by soft tissue descent of the portion of the soft tissue facial envelope being treated.

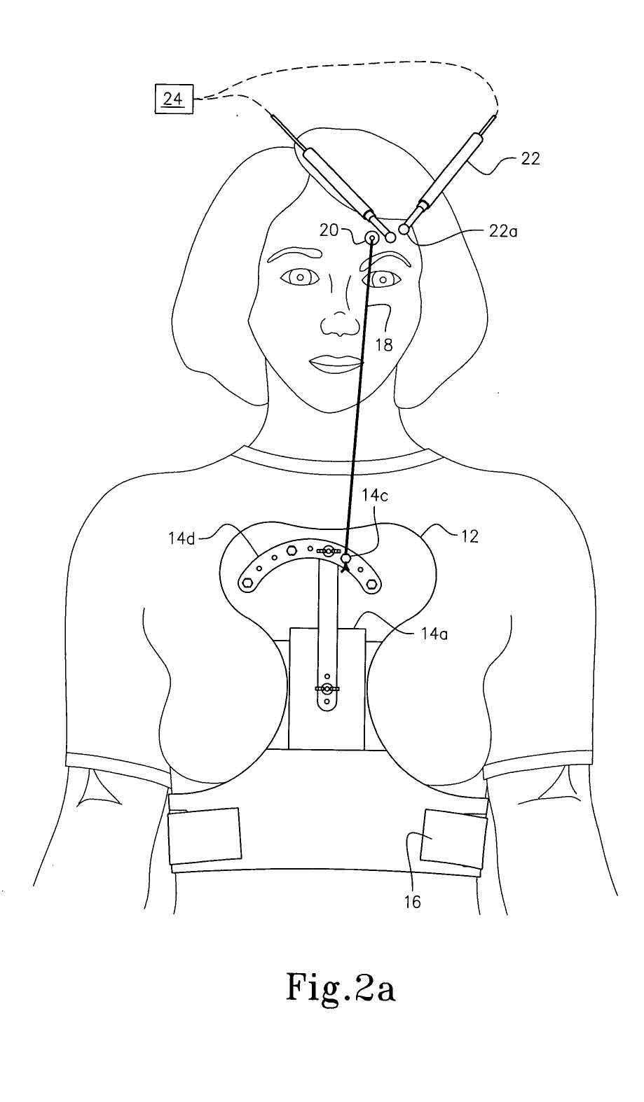

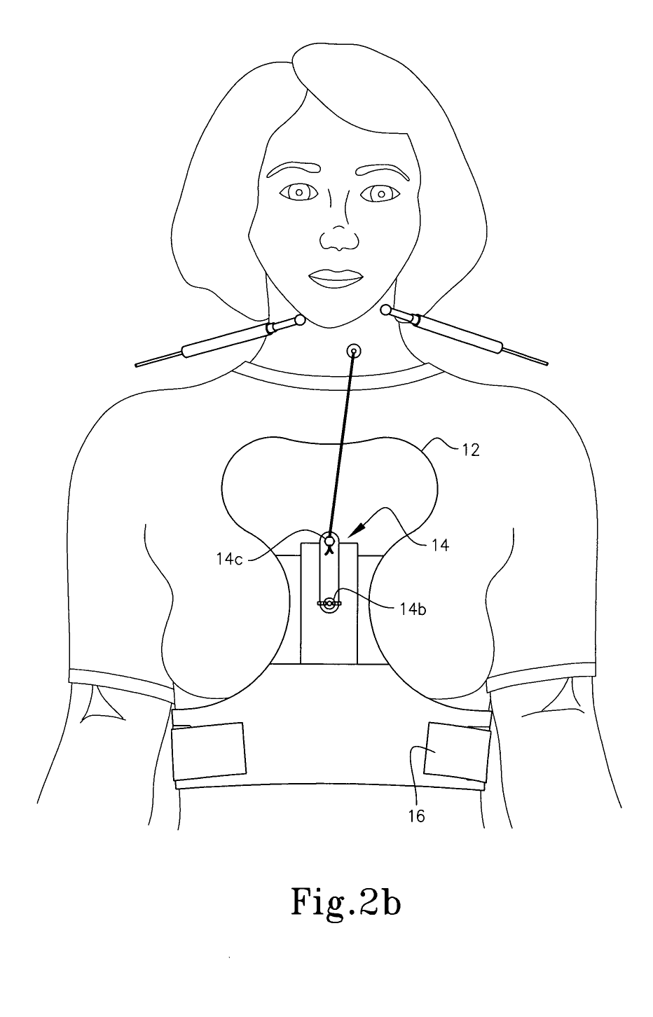

Referring to FIGS. 1, 2a and 2b, the apparatus or system 10 includes two electrode probes 22 adapted to be in electrical communication with means 24 for generating a desired current through the electrode probes 22 and for initiating an automated premodulated electrical current waveform which forces the contraction of the underlying muscle being treated under the tensile load.

The resistive outrigger 14 further comprises means 14a for aligning the resistive outrigger 14 so as to provide a non-interfering alignment of the elastomer bands 18 when treating the soft tissue facial envelope. The means 14a for aligning the resistive outrigger 14 is preferably a structural member (such as a roll-shaped structure) releasably attached to the generally planar-shaped fixation structure 12 to maintain said resistive outrigger 14 spaced-apart from the generally planar-shaped fixation structure 12.

The resistive outrigger 14 is adjustable for adding additional tensile load of the elastomer resistive bands 18 without re-securing the elastomer resistive bands 18 by rotating said resistive outrigger 14 about a pivot point 14b. The resistive outrigger 14 is preferably pivotally connected at one end to the means 14a for aligning the resistive outrigger 14 and is typically made from a flat plate about 5/8 inch to about 3/4 inch in width and about 6 inches in length for the outrigger used for the facial treatment and about 3 inches length for the outrigger used for the neck treatment. Both forms of the outrigger are somewhat Z-shaped so the outrigger 14 can be radially rotated to further adjust tensile load from that originally obtained when the elastomer bands 18 were first set. Although each elastomer band 18 could be connected to an aperture in the outrigger 14, it is preferred that a device such as a plastic or nylon nut (see 14c) be threadly engaged to the outrigger 14. This nut 14c could have a dome-type top with an aperture bored therethrough, through which the elastomer band 18 can be inserted and secured. Typically, the shorter outrigger 14 used for the neck area has a one band 18 connection point, although it could have a lateral cross-member to form a generally T-shaped outrigger 14, such the configuration preferred for the treatment of the facial muscles.

This T-shaped outrigger 14 has a leg which is rotatably attached on one end (see pivot point 14b) to the structural member 14a attached to the generally planar-shaped fixation structure 12 and a cross-member 14d forming the T-shaped resistive outrigger 14. The cross-member 14d preferably has two or more spaced-apart resistive elastomer band attachment means 14c for selectively securing one end of the elastomer resistive bands to the outrigger 14. Like the shorter outrigger 14 described above, the attachment means 14c for the bands 18 may be a threaded nut with a bored through dome portion through which the elastomer band may be looped and secured.

As mentioned above, the present invention includes a novel design in the configuration or shape of the probe 22 tips 22a. That is, the tips 22a are spherical-shaped. It was found that an optimal range for effective administration of current was a probe 22 which has a tip 22a radius of about 0.15 inches to about 0.25 inches, and more preferably about 0.19 inches.

It was found that the radius of the probe 22 and the convex contact that is created is the optimal design configuration for delivering enough current to facilitate the contraction of the underlying muscle that is under a tensile load. It is also the optimal configuration to minimize burns at the margins of the radius as there is no sharp edges that might lead to current concentration points. A smaller radius would lead to the risk of electrical burns and a larger radius decreases the current density and reduces the full contractile excursion of the muscle fibers under stretch and then the efficacy of the procedure.

The concave shape and radius diminishes the risk of focal regions of electrical current density excess which would lead to thermal injuries at the interface of the probe to the skin.

The design of the probes 22 leads to great anatomic procession as the probes 22 may be moved over the skin slightly at the skin interface and new underlying muscles can be localized for contraction which will facilitate the contraction of that particular muscle. This precision allows for isolation of only those muscles of facial elevation and not the wrinkle muscles or muscles which pull the face down. Further, the probe radius and convex configuration optimizes the current density that safely enters the skin and minimizes the risks of burns in doing so.

The fixation structure 12 and the means 14a for aligning the resistive outrigger 14 are typically made of easily cleaned, flexible material such as polymer composite materials.

### METHOD OF USE

The present invention includes a method for the non-surgical soft-tissue contouring and elevation of the facial envelope including the brow, lids, midface, perioral region, jowls, cheeks and neck, by strengthening the facial elevators of said soft tissue facial envelope, that is, resistance training for those muscles specific to facial soft tissue skin, the method comprises:
inducing skeletal muscle hypertrophy by applying a series of applications of a tensile load to targeted facial envelope muscles of a patient while delivering a sufficient level of electrical current using electrode probes, whose tips have been pre-coated in a conductive gel, in contact with and located across said targeted facial envelope muscles to facilitate contraction of the underlying muscles under the tensile load,
wherein the tensile load creates a concentric and eccentric load on the muscle fibers of the targeted facial envelope muscles, and
wherein muscle fiber hypertrophy is optimized.

The apparatus or system 10 used for the above procedure is that described above. Detailed procedures and current (amperage) levels for each area treated, are discussed above, particularly, in the sections discussing Resistive Hypertrophy Techniques.

It should be understood that the preceding is merely a detailed description of one or more embodiments of this invention and that numerous changes to the disclosed embodiments can be made in accordance with the disclosure herein without departing from the spirit and scope of the invention. The preceding description, therefore, is not meant to limit the scope of the invention. Rather, the scope of the invention is to be determined only by the appended claims and their equivalents.

Now that the invention has been described,

1. An apparatus for producing facelift-like effects on the soft tissue facial envelope, including the brow, lids, midface, perioral region, jowls, cheeks and neck, by strengthening the facial elevators of said soft tissue facial envelope, the apparatus comprising:
a tensile resistive patient fixation structure;
a resistive outrigger releasably attachable to said fixation structure;
means for attaching said fixation structure to the torso of the patient;
one or more resistive elastomer bands;
one or more resistive elastomer band fixation tabs, said tabs being for releasable attachment to locations on the patient's soft-tissue to be treated using the apparatus; and
each elastomer resistive band being attachable at one end to the resistive outrigger and an opposite end of the elastomer resistive band being attachable to the fixation tab in such a way as to create a tensile load condition measured by soft tissue descent of the portion of the soft tissue facial envelope being treated.

2. The apparatus according to claim 1, further comprising:
two electrode probes; and
means for generating a desired current through the electrode probes and for initiating an automated premodulated electrical current waveform which forces the contraction of the underlying muscle being treated under the tensile load.

3. The apparatus according to claim 1, wherein the fixation structure is generally planar-shaped and configured to be in an overlying relationship with the front torso of the patient.

4. The apparatus according to claim 3, wherein the resistive outrigger is releasably attached to the fixation structure with mating hook and loop fastener means.

5. The apparatus according to claim 1, wherein the means for attaching said patient fixation structure to the torso of the patient includes belt means for securing the fixation means around the torso of the patient being treated.

6. The apparatus according to claim 5, wherein the belt means is securable using mating hook and loop fastener means.

7. The apparatus according to claim 3, wherein the resistive outrigger further comprises:
means for aligning the resistive outrigger so as to provide a non-interfering alignment of the elastomer bands when treating the soft tissue facial envelope; and
said means for aligning the resistive outrigger being a structural member releasably attachable to the generally planar-shaped fixation structure to maintain said resistive outrigger spaced-apart from the generally planar-shaped fixation structure.

8. The apparatus according to claim 2, wherein each probe further comprises:
a spherical-shaped tip.

9. The apparatus according to claim 8, wherein each probe has a tip radius of about 0.15 inches to about 0.25 inches.

10. The apparatus according to claim 9, wherein each probe has a tip radius of about 0.19 inches.

11. The apparatus according to claim 7, wherein the resistive outrigger is adjustable for adding additional tensile load of the elastomer resistive bands without re-securing the elastomer resistive bands by rotating said resistive outrigger about a pivot point.

12. The apparatus according to claim 11, wherein the resistive outrigger is generally T-shaped with a leg of said T-shaped resistive outrigger being rotatably attached on one end to the structural member attached to the generally planar-shaped fixation structure and a cross-member forming the T-shaped resistive outrigger, the cross-member having two or more spaced-apart resistive elastomer band attachment means for selectively securing one end of each of the elastomer resistive bands to the resistive outrigger.

13. Electrode probes for use with an apparatus for producing facelift-like effects on the soft tissue facial envelope consisting of the neck, jowls, cheeks and brow, by strengthening the facial elevators of said soft tissue facial envelope, the electrode probes having a spherical-shaped tip having a tip radius of about 0.15 inches to about 0.25 inches.

14. The electrode probes according to claim 13, wherein the electrode probes are in electrical communication with means for generating a desired current through said electrode probes and for initiating an automated premodulated electrical current waveform which forces the contraction of the underlying muscle being treated under a tensile load.

15. The electrode probes according to claim 13, wherein each probe has a tip radius of about 0.19 inches.

16. A method for non-surgical soft-tissue contouring and elevation of the facial envelope including the brow, lids, midface, perioral region, jowls, cheeks and neck, by strengthening the facial elevators of said soft tissue facial envelope, that is, resistance training for those muscles specific to facial soft tissue skin, the method comprising:
inducing skeletal muscle hypertrophy by applying a series of applications of a tensile load to targeted facial envelope muscles of a patient while delivering a sufficient level of electrical current using electrode probes in contact with and located across said targeted facial envelope muscles to facilitate contraction of the underlying muscles under the tensile load,
wherein the tensile load creates a concentric and eccentric load on the muscle fibers of the targeted facial envelope muscles, and
wherein muscle fiber hypertrophy is optimized.

17. The method according to claim 16, wherein a system is provided for use in said method, the system comprising:
a tensile resistive patient fixation structure;
a resistive outrigger releasably attached to said fixation structure;
means for attaching said fixation structure to the torso of the patient;
one or more resistive elastomer bands;
one or more resistive elastomer band fixation tabs, said tabs being for releasable attachment to locations on the patient's soft-tissue to be treated using the apparatus;
each elastomer resistive band being attached at one end to the resistive outrigger and an opposite end of the elastomer resistive band being attached to the fixation tab in such a way as to create a tensile load condition measured by soft tissue descent of the portion of the soft tissue facial envelope being treated;
two electrode probes; and
means for generating a desired current through the electrode probes and for initiating an automated premodulated electrical current waveform which forces the contraction of the underlying muscle being treated under the tensile load.

18. The method according to claim 17, wherein the fixation structure is generally planar-shaped and configured to be in an overlying relationship with the front torso of the patient.

19. The method according to claim 17, wherein the resistive outrigger is releasably attached to the fixation structure with mating hook and loop fastener means.

20. The method according to claim 17, wherein the means for attaching said patient fixation structure to the torso of the patient includes belt means for securing the fixation means around the torso of the patient being treated.

21. The method according to claim 20, wherein the belt means is secured using mating hook and loop fastener means.

22. The method according to claim 18, wherein the resistive outrigger further comprises:
means for aligning the resistive outrigger so as to provide a non-interfering alignment of the elastomer bands when treating the soft tissue facial envelope; and
said means for aligning the resistive outrigger being a structural member releasably attached to the generally planar-shaped fixation structure to maintain said resistive outrigger spaced-apart from the generally planar-shaped fixation structure.

23. The method according to claim 17, wherein each probe further comprises:
a spherical-shaped tip.

24. The method according to claim 23, wherein each probe has a tip radius of about 0.15 inches to about 0.25 inches.

25. The method according to claim 24, wherein each probe has a tip radius of about 0.19 inches.

26. The method according to claim 22, wherein the resistive outrigger is adjustable for adding additional tensile load of the elastomer resistive bands without re-securing the elastomer resistive bands by rotating said resistive outrigger about a pivot point.

27. The method according to claim 26, wherein the resistive outrigger is generally T-shaped with a leg of said T-shaped resistive outrigger being rotatably attached on one end to the structural member attached to the generally planar-shaped fixation structure and a cross-member forming the T-shaped resistive outrigger, the cross-member having two or more spaced-apart resistive elastomer band attachment means for selectively securing one end of each of the elastomer resistive bands to the resistive outrigger.
